(19) United States
(12) Patent Application Publication
(10) Pub. No.: US 2011/0076254 A1
(43) Pub. Date: Mar. 31, 2011

(54) POROUS SCAFFOLDS FOR STEM CELL RENEWAL

(75) Inventors: Miqin Zhang, Bothell, WA (US); Zhensheng Li, Frederick, MD (US); Matthew Chi-hang Leung, Seattle, WA (US)

(73) Assignee: UNIVERSITY OF WASHINGTON, Seattle, WA (US)

(21) Appl. No.: 12/892,720

(22) Filed: Sep. 28, 2010

Related U.S. Application Data
(60) Provisional application No. 61/246,442, filed on Sep. 28, 2009.

(51) Int. Cl. A61K 35/12; C12N 5/0735; A61P 3/10; A61P 9/00; A61P 25/16; A61P 25/28; A61P 19/02; A61P 35/02

(52) U.S. Cl. 424/93.7; 435/395; 435/366; 435/354; 435/353

(57) ABSTRACT

A method for expanding a population of stem cells using a porous scaffold, a porous scaffold populated with renewed stem cells, methods of administering stem cells using the porous scaffold and cells collected from the porous scaffold, and methods for tissue engineering and treating a condition treatable by administration of stem cells using the porous scaffold and cells collected from the porous scaffold.

### CROSS-REFERENCE TO RELATED APPLICATION

This application claims the benefit of U.S. Patent Application No. 61/246,442, filed Sep. 28, 2009, expressly incorporated herein by reference in its entirety.

### BACKGROUND OF THE INVENTION

Human embryonic stem cells (hESCs) have attracted a great deal of research interest because they have the regenerative capability to potentially produce any tissue in the body. The success of stem cell therapy is, however, dependent on the ability to reproducibly generate a large number of hESCs with high purity and consistency while maintaining their pluripotency.

Human embryonic stem cells (hESCs) are routinely cultured on fibroblast feeder layers or in fibroblast-conditioned medium, which requires continued supply of feeder cells and poses the risks of xenogenic contamination and other complications such as feeder-dependent outcome. Self-renewal of hESCs is commonly achieved by culturing hESCs on a feeder layer of mouse embryonic fibroblasts (MEFs) or human fibroblast feeder cells (hFFs) or in conditioned media (CM) derived from MEFs. The feeder layer provides a suitable substrate for hESC attachment, and releases nutrients and signaling factors, conditioning the media for maintenance of hESC pluripotency. Because MEFs or hFFs are viable for only a few days, the hESCs must be transferred regularly onto new feeder layers for continued renewal, which is a costly and labor-intensive procedure. Importantly, viruses or other undesired macromolecules from feeder cells may be transmitted to the hESC population and ultimately to the recipient of stem cell based therapy.

Although researchers have not come to a consensus on the exact governing mechanisms of stem cell self-renewal, studies have shown that the interaction between the ECM and the integrins on hESC membranes plays a critical role.

The ECM is believed to play a major role in stem cell renewal, in which ECM components either function as signaling molecules through integrin receptors or increase the sensitivity of cytokine receptors on stem cells. The use of ECM proteins as coating materials on substrates for stem cell renewal has been recently pursued. However, the matrices coated with proteins face the challenge of continued support for long-term cell function, as these coating materials can be depleted quickly over time. Furthermore, protein-based materials need to overcome the challenges of source-dependent variation, potential immune rejection, and infection by human and nonhuman pathogens.

Despite the advances in methods for stem cell renewal, a need exists for improved stem cell renewal methods. The present invention seeks to fulfill this need and provides further related advantages.

### SUMMARY OF THE INVENTION

The present invention provides a method for expanding a population of stem cells using a porous scaffold, a porous scaffold populated with renewed stem cells, methods of administering stem cells using the porous scaffold and cells collected from the porous scaffold, and methods for tissue engineering and treating a condition treatable by administration of stem cells using the porous scaffold and cells collected from the porous scaffold.

In one aspect, the invention provides a method for expanding a population of stem cells. In one embodiment, the method comprises seeding a porous scaffold (e.g., a porous scaffold comprising chitosan) with stem cells and renewing the stem cells in the presence of the scaffold to provide a scaffold populated with renewed stem cells. Suitable stem cells include embryonic stem cells and non-embryonic stem cells (adult or somatic stem cells). Representative stem cells include hematopoietic stem cells, bone marrow stem cells, neural stem cells, epithelial stem cells, skin stem cells, muscle stem cells, and adipose stem cells. The stem cells can be human stem cells, mouse stem cells, or rat stem cells. In one embodiment, the renewed stem cells substantially maintain the pluripotency of the seeded stem cells. In one embodiment, the renewed stem cells are substantially undifferentiated.

In one embodiment, the scaffold comprises a chitosan. In one embodiment, the scaffold further comprises an alginate. In one embodiment, the scaffold comprises chitosan ionically linked to alginate further crosslinked with divalent metal cations. In one embodiment, the scaffold further comprises a fibrous protein. In certain embodiments, the scaffold further comprises one or more growth factors.

In one embodiment, renewing the stem cells comprises proliferation in the absence of feeder cells. In one embodiment, renewing the stem cells comprises proliferation in the absence of conditioned media.

In another aspect of the invention, a porous scaffold (e.g., a porous scaffold comprising chitosan) populated with renewed stem cells prepared by the method of the invention.

In a further aspect, the invention provides a method for administering stem cells to a subject, comprising introducing the porous scaffold populated with renewed stem cells into a subject in need thereof. Suitable stem cells include hematopoietic stem cells, bone marrow stem cells, neural stem cells, epithelial stem cells, skin stem cells, muscle stem cells, and adipose stem cells. In one embodiment, the subject is a human.

In another aspect, the invention provides a method for tissue engineering, comprising introducing the porous scaffold populated with renewed stem cells into the tissue to be engineered.

In a further aspect, the invention provides a method for treating a condition treatable by the administration of stem cells. In one embodiment, the method includes introducing the porous scaffold populated with renewed stem cells into a subject in need thereof. In another embodiment, the method includes seeding a porous scaffold with stem cells, renewing the stem cells in the presence of the scaffold to provide a scaffold populated with renewed stem cells, collecting at least a portion of the renewed stem cells from the scaffold, and administering renewed stem cells collected from the scaffold to a subject in need thereof. In certain embodiments of the above methods, the subject in need thereof suffers from a condition selected from the group consisting of diabetes, heart diseases, Parkinson's disease, Alzheimer's disease, arthritis, leukemias, lymphomas, and bone marrow failure disorders.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings.

FIG. 1A is an image of a representative scaffold (chitosan-alginate, CA) as synthesized and a scaffold section useful in the method of the invention for stem cell renewal. FIG. 1A is a scanning electron microscope (SEM) image illustrating the porous structure of the representative scaffold illustrated in FIG. 1A. FIG. 1C is a graph illustrating pore size distribution of the representative scaffold illustrated in FIG. 1A assessed by mercury porosimetry.

FIGS. 2A and 2B compare in vitro proliferation and pluripotency of human embryonic stem cells (hESCs) in a representative scaffold with hESCs grown on human fibroblast feeder cell (hFF) layers as controls. FIG. 2A compares cell proliferation as a function of time by alamarBlue assay; the hESCs were proliferated in the scaffold without subculturing for 21 days, and the hESCs on hFF layers were subcultured every 6 days. FIG. 2B compares alkaline phosphatase (ALP) activity as a function of cell culture time for hESCs in a representative scaffold (B) and for hESCs grown on hFF layers.

FIG. 3 is a bar graph comparing in vitro pluripotency of hESCs in scaffolds with hESCs grown on hFF layers as controls as a function of time (7, 14, and 21 days). Gene activity was assessed by RT-PCR. The values are presented as relative to the expressions by hESCs cultured on hFF layers, and normalized against β-actin expression. All the results are expressed as the mean±standard deviation.

FIGS. 4A-4F presents images of the undifferentiated state of hESCs assessed by immunological detection of SSEA 4 and cell morphology. FIGS. 4A and 4B are images of hESCs grown in scaffolds stained with DAPI (blue) and SSEA 4 antibody (green) showing cell localization and SSEA4 expression. FIG. 4C is the overlay of FIGS. 4A and 4B. Scale bars are 40 μm for FIGS. 4A-4C. FIG. 4D is the overlay image of FIG. 4C at higher resolution revealing the details of the co-localization of SSEA4 and cells. Scale bar is 10 μm for FIG. 4D. FIGS. 4E and 4F are SEM images of hESCs grown within the porous structure of CA scaffolds: FIG. 4F is an SEM image at magnification greater than that of the image in FIG. 4E showing cell morphology and cluster structure. Scale bar is 50 μm for FIG. 4E and the scale bar is 10 μm for FIG. 4F. FIGS. 4G and 4H compare the flow cytometric results for the SSEA4 activity of hESCs harvested from scaffolds (21 days) and on hFF layers (7 days), respectively.

FIGS. 5A-5F presents histological analysis of teratomas retrieved after one month implantation of hESC-scaffold constructs in SCID mice. FIG. 5A is an image of an explanted teratoma (scale bar: 1 cm). FIG. 5B is an image of a dissected teratoma showing nodules of tissue (scale bar: 1 cm). FIG. 5C is an image of the explant stained with Von Kossa showing islands of calcification (black) in the center of the specimen indicating initial bone formation (scale bar: 50 μm). The calcification suggests that mono-nucleated cells in the center may be osteogenic cell types. FIG. 5D is an image of the explant stained with Picrosirius for collagen (red) and cells (dark grey) indicating the formation of dense collagen and cell-lined lumens that are similar to secretory linings (seen in other ductular tissue) (scale bar: 50 μm). FIG. 5E is an image of the explant stained with Picrosirius red showing formation of blood vessels and clusters of large polygonal cells that resemble adipocytes and hepatocytes (scale bar: 100 μm). FIG. 5F is an image of the explant stained with silver showing formation of cross-striated muscle with characteristic pattern of striations (scale bar: 100 μm).

FIG. 6 presents images assessing in vivo pluripotency of hESCs cultured in scaffolds for three weeks. The explants were harvested from SCID mice 1 month after implantation. Tissue sections were DAPI-stained (second column) for nuclei and immunocytochemically stained (first column) for cardiac troponin, smooth muscle actin, FOXA2, glucagon, and NCAM lineage markers. The third column overlays images from the first and second columns. Scale bars are 20 μm.

FIG. 7 is a graph comparing hESCs proliferation in a representative scaffold without subculturing as a function of time for two different passages by alamarBlue assay.

FIGS. 8A-8C compare flow cytometric results assessing the undifferentiated state of hESC by immunological detection of SSEA 4 expression. FIG. 8A illustrates SSEA 4 expression of hESCs harvested from hFF layers after 7 days of culture. FIG. 8B illustrates SSEA 4 expression of hESCs recovered from scaffolds after 14 days of culture. FIG. 8C illustrates SSEA 4 expression of hESCs recovered from scaffolds after 14 days of culture and subcultured for an additional 14 days.

### DETAILED DESCRIPTION OF THE INVENTION

The present invention provides methods for stem cell renewal using porous, three-dimensional scaffolds that effectively support self-renewal of hESCs without the need of feeder cell layers or conditioned media, thereby eliminating potential biological contamination. The porous three-dimensional scaffold with the combined material properties and structural advantages provides cues that promote adhesion and proliferation of stem cells without feeder layers in unconditioned culture media, potentially mimicking the three-dimensional structure of native tissue. Due to biocompatibility and biodegradability of the scaffolds, hESCs populated scaffolds can be directly implanted for additional in vivo structural support for tissue engineering. The results from the method of the invention demonstrate that the renewed stem cells can be readily harvested at a high yield for subculture. The method avoids the problems associated with using trypsin or collagenase to release cells including the adverse effect on cell function, the difficulty in detaching cells from three-dimensional scaffolds resulting in a low cell yield, and the inability to obtain hESC embryoid bodies.

In one aspect, the invention provides a method for expanding a population of stem cells. In the method, a porous scaffold is seeded stem cells and the stem cells in the presence of the scaffold (e.g., in contact with the scaffold) are renewed to provide a scaffold populated with renewed stem cells. In the method, at least a portion of the seeded stem cells are in contact with the scaffold's surface (e.g., situated in the scaffold's pores).

As used herein the phrase “expanding a population of stem cells” refers to proliferating a population of stem cells to provide an expanded population (i.e., larger number) of stems cells that substantially maintain the parent stem cells' pluripotency (e.g., substantially maintain the parent stem cells' level of undifferentiation). In the practice of the invention, seeded stem cells are renewed in the presence of the porous scaffold. Stem cell renewal involves the maintenance of the cell's pluripotency under continued proliferation. As used herein, the term “stem cell expansion” is used interchangeably with the “stem cell renewal.”

The porous scaffold advantageously supports stem cell renewal. Suitable scaffolds have a porosity of from about 85 to about 96 percent. In one embodiment, the scaffold has a porosity of from about 91 to about 95 percent. In another embodiment, the scaffold has a porosity of from about 94 to about 96 percent. Suitable scaffolds have an average pore size diameter of from about 50 to about 200 μm. In one embodiment, the scaffold has an average pore size diameter of from about 40 to about 90 μm. In another embodiment, the scaffold has an average pore size diameter of from about 60 to about 150 μm. In one embodiment, the scaffold has a porosity of from about 85 to about 96 percent and an average pore size diameter of from about 50 to about 200 μm.

The porous scaffold possesses mechanical strength. The scaffold has a compressive yield strength of at least 0.35 MPa. In one embodiment, the scaffold has a compressive yield strength of from about 0.35 MPa to about 0.5 MPa. The scaffold has a compressive modulus of from about 5 MPa to 8 MPa. In one embodiment, the scaffold has a compressive yield strength of from about 0.35 MPa to about 0.5 MPa and a compressive modulus of from about 5 MPa to 8 MPa.

In one embodiment, the scaffold has a porosity of from about 85 to about 96 percent, an average pore size diameter of from about 50 to about 200 μm, a compressive yield strength of from about 0.35 MPa to about 0.5 MPa, and a compressive modulus of from about 5 MPa to 8 MPa.

The porous scaffolds of the invention include one or more natural polymers. As used herein, the term “natural polymer” refers to a polymer found in nature, excluding synthetic polymers, that can be formed into a scaffold having the porosity and pore size distribution described above. In one embodiment, the invention provides a porous scaffold comprising one or more natural polymers. Suitable natural polymers include extracellular matrix (ECM) proteins. In one embodiment, the invention provides a porous scaffold comprising one or more extracellular matrix (ECM) proteins. Suitable natural polymers also include proteoglycans or glycosaminoglycans. In one embodiment, the invention provides a porous scaffold comprising one or more proteoglycans or one or more glycosaminoglycans. The porous scaffold of the invention can include one or more fibrous proteins, such as collagens and laminins.

In one embodiment, the porous scaffold is a porous scaffold comprising chitosan. In this embodiment, the scaffold is made from a chitosan. Chitosans are linear polysaccharides composed of randomly distributed β-(1-4)-linked D-glucosamine (deacetylated unit) and N-acetyl-D-glucosamine (acetylated unit). Chitosans useful for making the scaffolds have an average molecular weight from about 10 kDa to about 1000 kDa. Generally, scaffolds made from higher molecular weight chitosans have greater mechanical strength than scaffolds made from lower molecular weight chitosans. An exemplary range of percentage deacetylation of chitosan useful for making the scaffolds is from about 80% to about 100% deacetylation.

In one embodiment, the porous scaffold comprises chitosan and alginate (i.e., a porous chitosan/alginate scaffold). Alginates are linear polysaccharides of β-D-mannuronic acid and α-L-guluronic acid. In these scaffolds, chitosan is ionically linked to alginate. As used herein, the term “ionically linked” refers to a non-covalent chemical bond or associative interaction between two ions having opposite charges (e.g., electrostatic association between a chitosan amine group and an alginate carboxylic acid group present on alginate).

Porous scaffolds comprising chitosan and alginate may be crosslinked to increase their mechanical strength. In one embodiment, the porous chitosan/alginate scaffold is crosslinked with divalent metal ions. Thus, in one embodiment, in addition to the ionic linkages between chitosan and alginate, the scaffolds include ionic linkages formed between alginate carboxylic acid groups and divalent metal ions (e.g., Ca²⁺, Ba²⁺, Mg²⁺, Sr²⁺). While not wishing to be bound by theory, it is believed that the divalent metal cations form ionic linkages between adjacent alginate chains, thereby ionically cross-linking adjacent alginate molecules.

In one embodiment, the scaffold further comprises one or more growth factors or inhibitory factors effective for stem cell renewal such as basic fibroblast growth factor (bFGF) and leukemia inhibitory factor.

In one embodiment, the scaffold further comprises one or more growth factors effective for stem cell differentiation. For example, growth factors (Activin-A and transforming growth factors TGFβ1) induce mesodermal cells and factors (retinoic acid, EGF, BMP-4) activate ectodermal and mesodermal markers and factors (nerve growth factor NGF and hepatocyte growth factor HGF) that allow differentiation into the three embryonic germ layers.

The preparation and characteristics of a representative scaffold are described below and in Example 1.

Stem cells suitable for renewal by the method of the invention include embryonic stem cells and non-embryonic stem cells (adult or somatic stem cells). Representative stem cells include hematopoietic stem cells, bone marrow stem cells (also known as mesenchymal stem cells or skeletal stem cells), neural stem cells, epithelial stem cells, skin stem cells, muscle stem cells, and adipose stem cells.

In one embodiment, the stem cells are human stem cells. In another embodiment, the stem cells are mouse stem cells. In a further embodiment, the stem cells are rat stem cells. The expansion of populations of mouse and rat stem cells is particularly useful for fundamental stem cell studies including animal studies.

As noted above, in certain embodiments of the method of the invention, the population of stem cells is expanded while substantially maintaining the pluripotency of the stem cells seeded into the scaffold. In certain embodiments, the population of stem cells is expanded without substantial differentiation (i.e., the renewed stem cells are substantially undifferentiated relative to the stem cells seeded into the scaffold). As used herein, “substantially maintaining the pluripotency of the stem cells seeded into the scaffold” refers to an expanded population of stem cells wherein at least about 90% of the cells maintain the pluripotency of the stem cells seeded into the scaffold. As used herein, the term “substantially undifferentiated” refers to a population of stem cells wherein at least about 90% of the cells are undifferentiated relative to the stem cells seeded into the scaffold. In the practice of the invention, stem cells can be expanded for a period of time up to about six months (i.e., the expanded population of stem cells maintain their pluripotency and/or are substantially undifferentiated).

Through the advantageous use of the porous scaffold and in contrast to conventional methods, the invention provides a method for stem cell renewal that does not require feeder cells. Conventional methods for stem cell renewal employ feeder cells. In these methods, the inner surface of culture vessel coated with mouse embryonic skin cells that have been treated so that they do not proliferate and that provides a feeder layer.

In the method of the invention, stem cells are renewed without conditioned media derived from the feeder layers with culture media, but in the absence of feeder cells. Scaffolds made of synthetic polymers, such as poly(lactic-co-glycolic acid) (PLGA), polylactic acid (PLA), and polycaprolactone (PCL), cannot be used to renew stem cells in the absence of feeder layers.

Thus, in one aspect, the invention provides a method for expanding a population of stem cells comprising seeding a porous scaffold, as described above, with stem cells and renewing the stem cells in the presence of the scaffold and in the absence of feeder cells. In one embodiment, the invention provides a method for expanding a population of stem cells that have hereto for required the use of a feeder cell layer to effect stem cell proliferation. In this embodiment, the method comprises seeding a porous scaffold of the invention as described above with stem cells and renewing the stem cells in the presence of the scaffold and in the absence of feeder cells.

In another aspect, the invention provides a scaffold populated with stem cells. In one embodiment, the scaffold is populated with renewed stem cells. The scaffold is prepared by the method of the invention described above. The scaffold does not include feeder cells. The scaffold comprising stem cells can be used to administer stem cells.

In another aspect of the invention, a method for administering stem cells is provided. In one embodiment, the method for administering stem cells comprises introducing a porous scaffold populated with stem cells to a subject in need thereof. Suitable subjects to which the porous scaffold populated with stem cells can be administered include warm-blooded animals, such as humans. The nature of the stem cells administered in not limited and includes those stem cells noted above.

The introduction of the porous scaffold populated with stem cells can be used for a variety of purposes including, for example, tissue engineering. For tissue engineering, the porous scaffold is introduced into the tissue to be engineered. For specific tissue regeneration, specific adult stem cells can be used. Embryonic stem cells can be differentiated into all types of tissues in the presence of growth factors for cell differentiation. Alternatively, as described below, renewed stem cells collected from the porous scaffold populated with renewed stem cells can be introduced.

In another aspect, the invention provides methods of treatment using the scaffold populated with stem cells. In one embodiment, the invention provides a method for treating a condition treatable by the administration of stem cells. In the method, the scaffold populated with stem cells is introduced into a subject in need thereof.

In another embodiment, the invention provides methods of treatment using the renewed stems collected from the porous scaffold populated with the stem cells. In one embodiment, the method includes seeding a porous scaffold with stem cells; renewing the stem cells in contact with the scaffold to provide a scaffold populated with renewed stem cells; collecting at least a portion of the renewed stem cells from the scaffold; and administering renewed stem cells collected from the scaffold to a subject in need thereof.

In the above methods, human embryonic stem cells can be used to treat spinal cord injury, diabetes, heart diseases, Parkinson's disease, Alzheimer's disease, and arthritis, among others. Human cord blood stem cells can be used to treat leukemias, lymphomas, and other blood cancers, and bone marrow failure disorders. T stem cells can be used to treat various cancers. Muscle and skeletal stem cells can be used to treat muscle and skeletal diseases or loss, respectively.

The following is a description of a representative porous scaffold and its use in renewing stem cells.

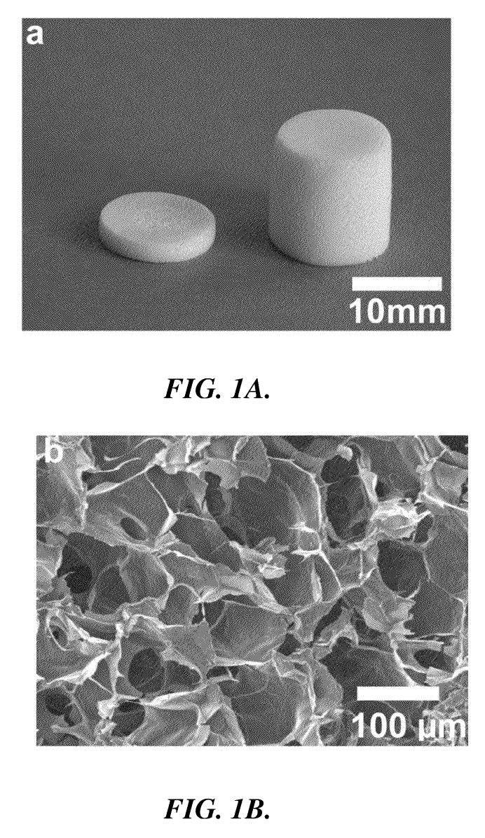

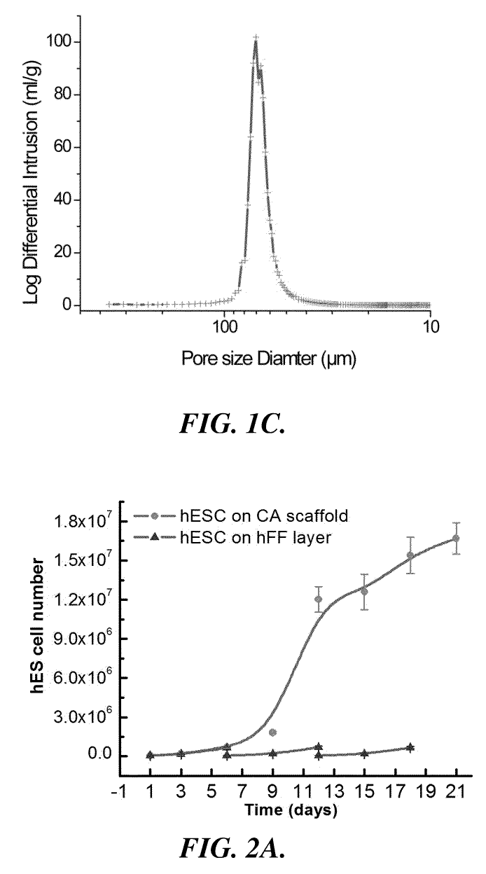

Synthesis and characterization of representative chitosan-alginate (CA) scaffolds. The porous structure of CA scaffolds was created through a process of thermally induced phase separation and subsequent solvent sublimation. The as-synthesized cylindrical scaffolds were cut into sections of 13 mm diameter and 2 mm thickness for subsequent in vitro studies (FIG. 1A). The CA scaffolds have a highly porous structure with interconnected pores, a porosity of about 95%, an average pore size of about 65 μm and a narrow size distribution. The compressive and tensile modules of the scaffolds are much higher than those of pure chitosan scaffolds due to strong ironic interaction of amine groups in chitosan with carboxyl groups in alginate providing a sustainable cell culture environment for hESC renewal in culture media. The preparation and characteristics of a representative scaffold are described in Example 1.

The ratio of chitosan to alginate in the scaffolds ranges from about 1:1 to about 4:1 (e.g., 1:1 to 3:1, or 1:1 to 2:1). The scaffolds are composed almost entirely of chitosan and alginate. Divalent metal ions are present in the scaffold typically in an amount less than about 1% by weight of the scaffold.

Representative scaffolds have a compressive yield strength from about 0.35 MPa to about 0.8 MPa, a compressive modulus from about 5 MPa to 8 MPa, and a porosity in the range of from 88% to 96%.

Suitable scaffolds can be made by freezing a solution comprising the chitosan and the alginate to produce a frozen structure, drying the frozen structure to produce a dried structure, and contacting the dried structure with divalent metal cations to provide the scaffold.

The ratio of chitosan to alginate in the solution is typically in the range of from 1:1 to 4:1. The concentration of alginate in the solution is typically in the range of from 1.5% (w/v) to 2.5% (w/v). The concentration of chitosan in the solution is typically in the range of from 1.5% (w/v) to 2.5% (w/v). The pH of the solution of chitosan and alginate is typically from 6.0 to 8.0. The solution of chitosan and alginate is typically frozen at a temperature of from −10° C. to −20° C. The frozen structure is dried, for example by lyophilization. The moisture content of the dried structure approaches zero (e.g., a moisture content of less than 0.5% by weight).

In the method, the dried structure is contacted with divalent metal cations by, for example, immersing the structure in a solution of divalent metal cations or spraying the structure with a solution of divalent metal cations. A useful, exemplary, concentration of divalent metal cations in a solution of divalent metal cations is about 1% w/v.

The freezing temperature used in the process affects the porosity, pore size, and pore distribution in the scaffolds. In general, scaffolds prepared at lower freezing temperatures exhibit smaller pores with a more uniform pore structure. In general, scaffolds prepared at higher freezing temperature have larger pores than scaffolds prepared at lower freezing temperature, and the shapes of the pores are heterogeneous. The molecular weights of the chitosan and alginate also affect pore size and structure. Typically, use of higher molecular weight chitosan and alginate produces scaffolds having smaller pores that are less interconnected compared to scaffolds produced using lower molecular weight chitosan and alginate.

Representative scaffolds useful in the practice of the invention are described in U.S. Pat. No. 7,736,669, expressly incorporated herein by reference in its entirety.

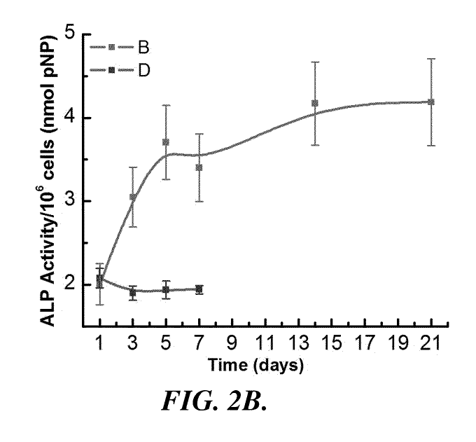

Cell proliferation and ALP activity. The self-renewal of hESC was initiated by directly seeding stem cells on CA scaffolds that were maintained in normal cell culture media. hESC proliferation in CA scaffolds was assessed by the alamarBlue assay and compared with hESCs cultured on hFF layers as a reference. Cell proliferation rates in both systems were comparable in the first 6 days. Thereafter, hESCs grown on hFF layers detached from the degrading hFF layers and needed to be transferred to new culture plates with fresh hFF layers every 6 days, while hESC in CA scaffolds continued to proliferate without subculturing for the entire culture period of 21 days (FIG. 2A). hESC proliferation in CA scaffolds was found to be exponential in the first 12 days and essentially linear thereafter. This result may be due to the initial rapid migration and expansion of the cells as they continually occupied the inner walls of the porous structure of the scaffold, after which further expansion of the cell population was confined to within the scaffold pores. In principle, the duration of the sustained proliferation is limited only by the dimensions of the scaffold and the diffusion parameters of nutrition and metabolic exchange between the culture medium and the interconnected scaffold pores.

The undifferentiated state of the hESCs was determined by alkaline phosphatase activity (ALP), gene activity, cell morphology, and expression of surface marker stage-specific embryonic antigen-4 (SSEA4). Alkaline phosphatase (ALP) is a characteristic biochemical marker of undifferentiated hESCs. The ALP activity (normalized to the cell number) of hESCs grown on CA scaffolds was measured over 21 days and compared with hESCs grown on hFF layers (FIG. 2B). The ALP activity of hESCs in CA scaffolds increased for the first week and reached a plateau after day 15 while the ALP activity of hESCs on hFF layers demonstrated a slight initial decrease and reached a plateau at day 3. The alkaline phosphatase activity of hESCs on chitosan-alginate scaffolds cultured for 7 days without feeder cells was 3 times higher than that of the hESCs co-cultured with feeder cells on tissue culture plates.

In vitro assessment of hESC pluripotency. The gene expression patterns of hESCs cultured in CA scaffolds and on hFF layers were quantified by real-time PCR (RT-PCR). These genes have been suggested as markers of undifferentiated hESCs or their differentiated derivatives. Among the thirteen genes evaluated, OCT 4, NANOG, TERT, TDGF 1, and REX-1 are known to be associated with the pluripotent state of hESCs. Alternatively, the lineage marker genes, including FOXA2, AFP, and Gluc for endoderm, Flk-1, ACTA2 (alpha smooth muscle actin), and TNNT2 (cardiac troponin) for mesoderm, and NCAM and SOX1 for ectoderm germ layer cells, are commonly known to be associated with differentiation of hESCs. Total RNA was harvested from hESCs grown in CA scaffolds every 7 days over a period of 21 days and from hESCs grown on hFF feeder layers for 7 days. The transcription levels of the thirteen representative genes were measured and their values are presented relative to the expressions by hESCs cultured on hFF, normalized against β-actin expression. The primer sequences used for RT-PCR are shown in Table 1.

TABLE 1
Primer sequences used for RT-PCR, grouped
according to relevance to hESC characterization.

| | | | |
|---|---|---|---|
| Gene | | | |
| symbol | Forward Primer | Reverse Primer | Gene name |

Undifferentiation markers

| | | | |
|---|---|---|---|
| OCT4 | 5′-CTT GCT GCA GAA GTG | 5′-CTG CAG TGT GGG TTT | Octamer-4, transcription factor |
| | GGT GGA GGA A | CGG GCA | |
| | (SEQ ID NO: 1) | (SEQ ID NO: 2) | |
| NANOG | 5′-CCT GAA CCT CAG CTA | 5′-TGC CAC CTC TTA GAT | nanOg, DNA regulatory protein |
| | CAA AC | TTC AT | |
| | (SEQ ID NO: 3) | (SEQ ID NO: 4) | |
| TERT | 5′-CGG AAG AGT GTC TGG | 5′-GGA TGA AGC GGA GTC | Telomerase reverse |
| | AGC AA | TGG A | transcriptase |
| | (SEQ ID NO: 5) | (SEQ ID NO: 6) | |
| TDGF1 | 5′-CAG GAA TTT GCT CGT | 5′-TAG TAC GTG CAG ACG | Teratocarcinoma-derived |
| | CCA TCT CGG | GTG GTA GTT | growth factor 1 precursor |
| | (SEQ ID NO: 7) | (SEQ ID NO: 8) | |
| REX1 | 5′-TGA AAG CCC ACA TCC | 5′-CAA GCT ATC CTC CTG | RNA-exonuclease 1 homolog |
| | TAA CG | CTT TGG | (S. cerevisiae)-like |
| | (SEQ. ID NO: 9) | (SEQ. ID NO: 10) | |

Endoderm markers

| | | | |
|---|---|---|---|
| FOXA2 | 5′-TGT TGC AGG GAA GTC | 5′-ATG GTT TTA CAC CGA | Forkhead box protein, |
| | TTA CT | GTC AC | transcription activator for liver |
| | (SEQ. ID NO: 11) | (SEQ ID NO: 12) | function |
| AFP | 5′-AAG CCA CAA ATA ACA | 5′-GTC TTC TCT TCC CCT | Alpha-1- fetoprotein precursor, |
| | GAG GA | GAA GT | serum protein |
| | (SEQ ID NO: 13) | (SEQ ID NO: 14) | |
| GLUC | 5′-GGA TCT GGC AGC GCC | 5′-TTT TCC CAT CCA TTG | Glucosylceramidase precursor, |
| | GCG AAG ACG AGC GG | TGG GAC | metabolic enzyme |
| | (SEQ. ID NO: 15) | (SEQ ID NO:16) | |

Mesoderm markers

| | | | |
|---|---|---|---|
| FLK-1 | 5′-ACC ACA GTC CAT GCC | 5′-TTC ACC ACC CTG TTG | Protein-tyrosine kinase |
| | ATC AC | CTG TA | receptor, vascular endothelial |
| | (SEQ ID NO: 17) | (SEQ ID NO: 18) | growth factor |
| ACTA2 | 5′-TGT GGC ATC CAC GAA | 5′-GGA GCA ATG ATC TTG | Actin, aortic smooth muscle |
| | ACT AC | ATC TTC A | |
| | (SEQ ID NO: 19) | (SEQ ID NO: 20) | |
| TNNT2 | 5′-AGG CGC TGA TTG AGG | 5′-ATA GAT GCT CTG CCA | Troponin T, cardiac muscle |
| | CTC AC | CAG C | |
| | (SEQ ID NO 21) | (SEQ ID NO: 22) | |

Ectoderm markers

| | | | |
|---|---|---|---|
| NCAM | 5′-CAA AAA GGT GGA TAA | 5′-CAG GTA AGA GTG ACC | Neural cell adhesion molecule |
| | GAA CG | TGC TC | |
| | (SEQ ID NO: 23) | (SEQ ID NO: 24) | |
| SOX1 | 5′-ATG CAC CGC TAC GAC | 5′-CTT TTG CAC CCC TCC | SOX-1 protein, transcription |
| | GTG A | CAT TT | activator for neural |
| | (SEQ ID NO: 25) | (SEQ ID NO: 26) | development |

Cell motility

| | | | |
|---|---|---|---|
| ACTB | 5′-TTA GTT GCG TTA CAC | 5′-AAT GTG CAA TCA AAG | Beta cytoskeletal actin |
| | CCT TT | TCC TC | |
| | (SEQ ID NO: 27) | (SEQ ID NO: 28) | |

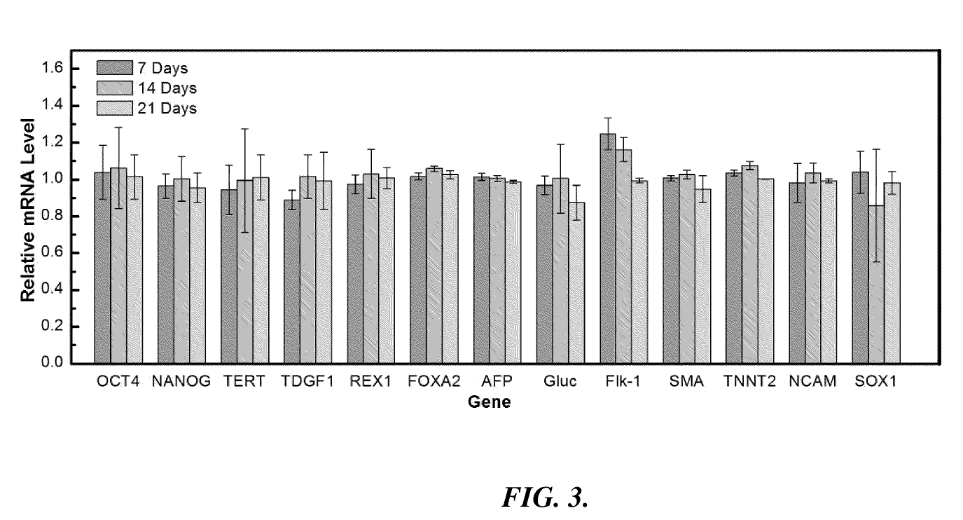

hESCs grown in CA scaffolds for 21 days expressed the five pluripotent marker genes (Oct 4, NANOG, TERT, TDGF1, and REX1) at the levels comparable to those expressed by hESCs grown on hFF layers for 7 days (FIG. 3). The continued expression of these marker genes suggests the persistence of pluripotent state of hESCs in CA scaffolds. The levels of lineage marker genes (FOXA2, AFP, Gluc, Flk-1, ACTA2, TNNT2, NCAM, and SOX1) expressed by hESCs grown in CA scaffolds were also similar to the levels expressed by those grown on hFF layers.

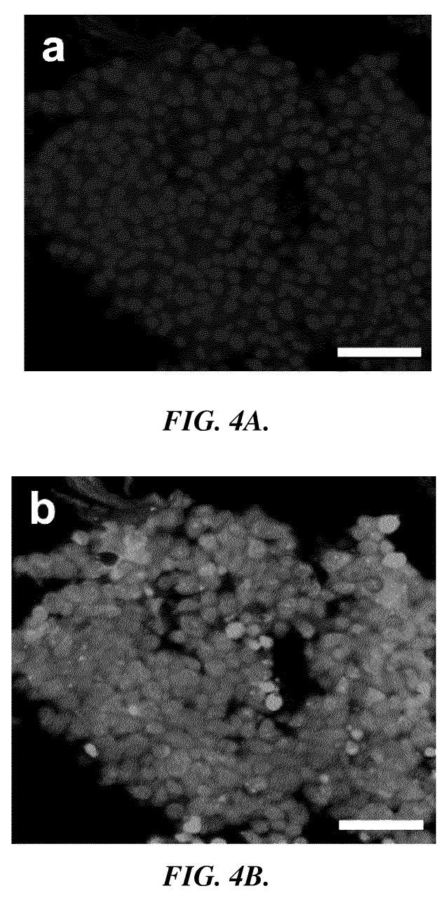

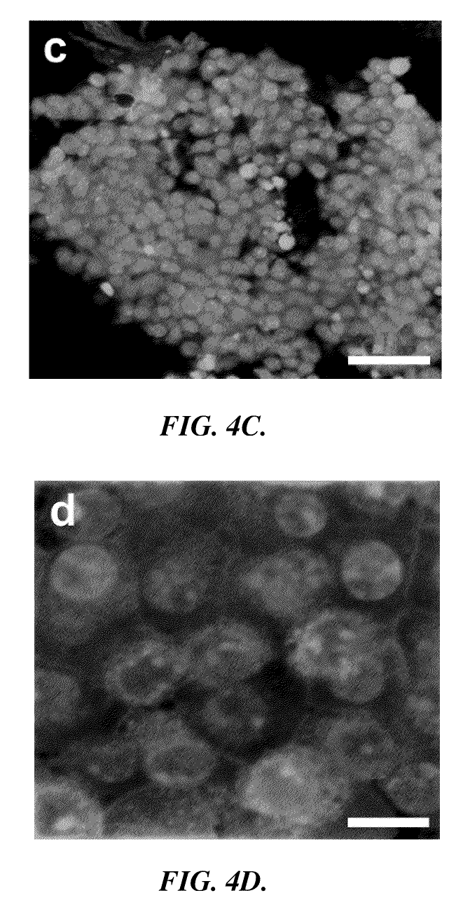

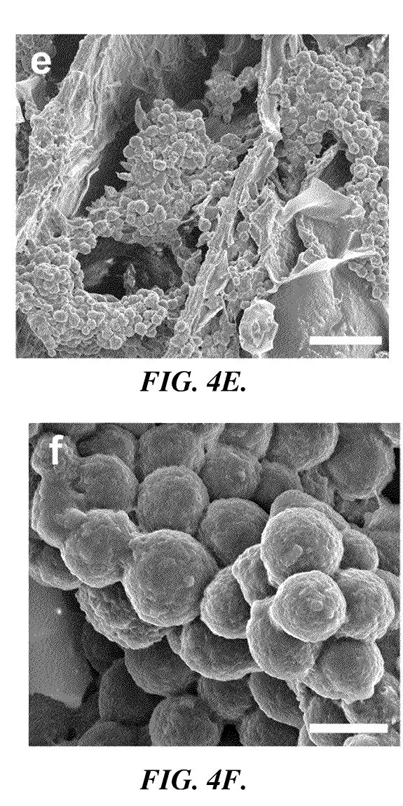

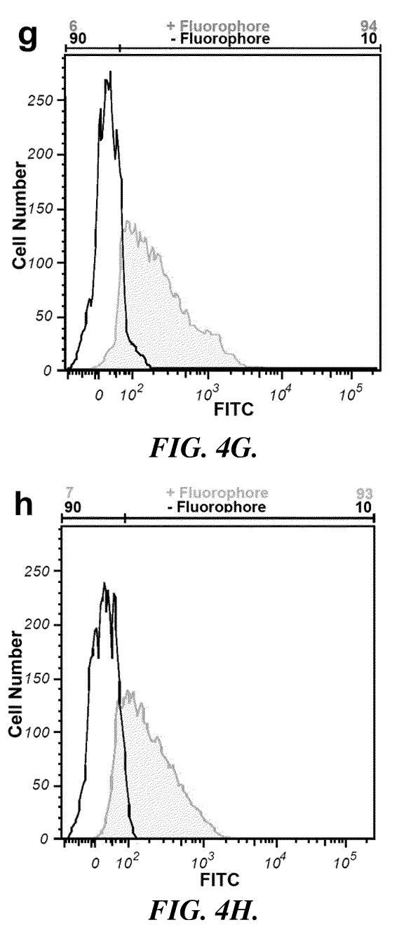

The undifferentiated state of hESCs was further assessed by immunological detection of the SSEA4, which is widely used as a surface marker of pluripotent stem cells and by cell morphology examination with SEM. After 21 days of culture in CA scaffolds, hESCs were stained with DAPI (FIG. 4A) and mouse anti-SSEA-4 antibody (FIG. 4B). The overlaid image (FIG. 4C) shows that the hESCs maintained SSEA4 expression and formed dense clusters in CA scaffolds. The image at higher magnification (FIG. 4D) shows no evidence of differentiated cells. Morphology of the hESCs in CA scaffolds was visualized using SEM. The cells were seen to form dense clusters of embryoid bodies on the pore walls within the scaffold (FIG. 4E) and the SEM image at higher magnification reveals that the cells exhibited a spherical shape that is the characteristic cell morphology of undifferentiated hESCs (FIG. 4F). The SSEA4 level (94%) expressed by hESC cells harvested from CA scaffolds was essentially equal to that expressed by hESCs grown on hFF layers, as quantified by flow cytometry (FIGS. 4G and 4H).

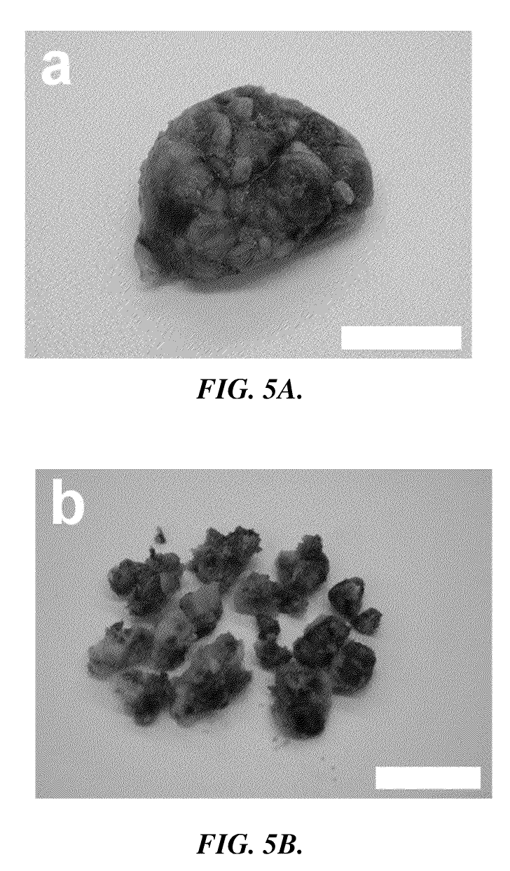

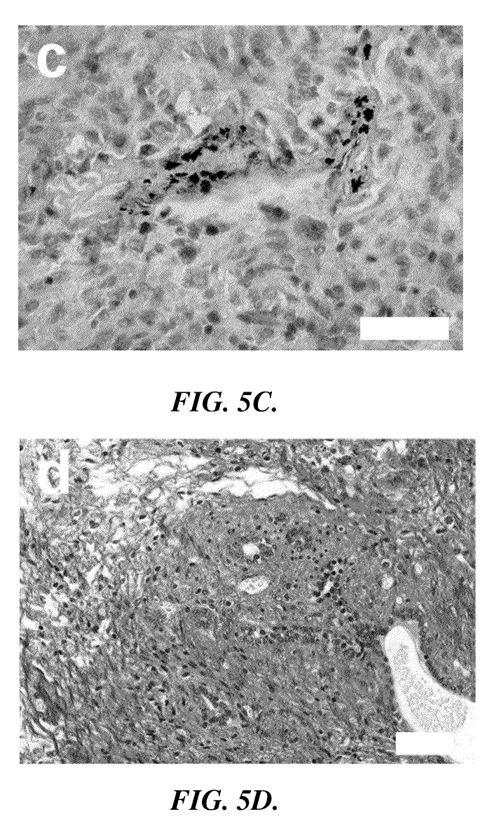

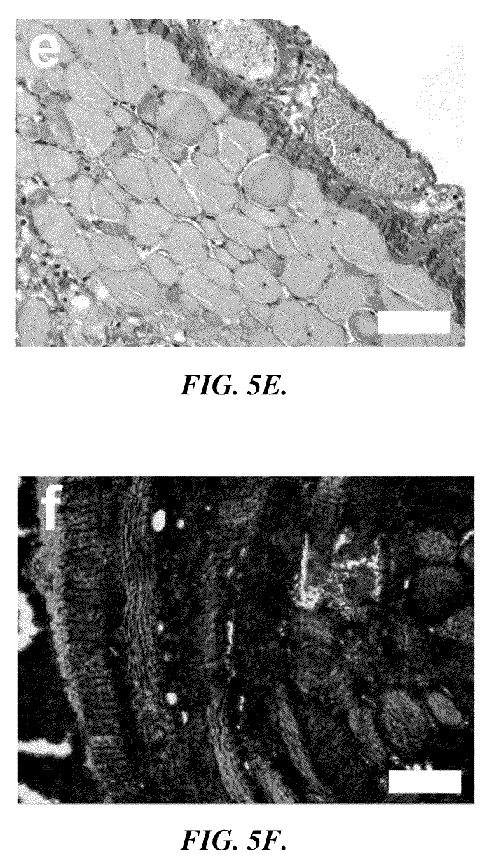

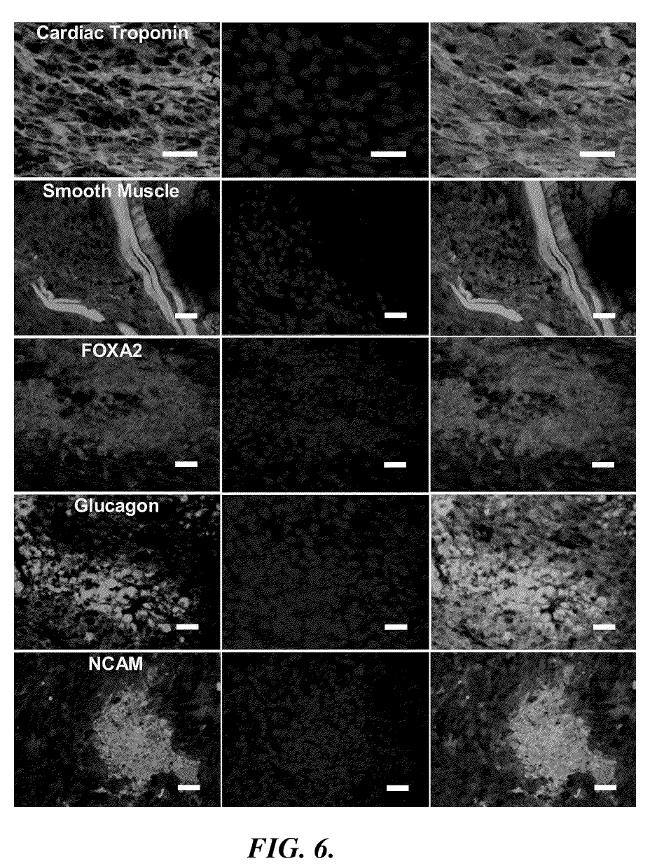

In vivo assessment of hESC pluripotency. To assess the potential of hESCs grown in CA scaffolds to form derivatives of all three embryonic germ layers (endoderm, mesoderm, and ectoderm), cells were cultured in CA scaffolds for 21 days and the cell-scaffold constructs of 3 mm×5 mm×5 mm were implanted into the abdominal cavities of SCID nude mice to induce cell differentiation and teratoma formation. The mice were sacrificed one month following implantation. The harvested teratomas had an average diameter of 15 mm, much larger than the original implants. Although the tumor was adherent to the surrounding tissues, no invasion of adjacent organs, such as intestine, liver, or peritoneal membrane, was observed. The teratoma was grossly heterogeneous, and histological images revealed the formation of mesodermal and endodermal types of tissues including calcified regions, collagen, blood vessels, and muscle tissue (FIGS. 5A-5F). Tissues of ectodermal lineage were not obvious in histological examination of H&E and Picrosirius red stained sections. Immunohistochemical fluorescent staining of teratoma sections was applied to confirm the formation of the three germ layers. Glucagon is a marker of the endoderm and has been used to study hESCs differentiated into pancreatic cells. Alpha smooth muscle actin, cardiac troponin-T, and FOXA 2 are all mesoderm tissue markers. NCAM was selected as a marker for detection of ectoderm formation. The confocal images show various tissues labeled with antibodies (FIG. 6). The first column displays antibody-labeled epitopes in either red or green color, indicating the presence of specific cells/tissues. The second column shows cell localization by nuclear staining. The third column is the overlay of the first and second columns providing the spatial relation between cell-specific epitopes and cell localization. The distinctive branched cellular network of cardiac tissue is visible with Cardiac Troponin stained samples (FIG. 6, Cardiac Troponin). The images also show striated muscles (FIG. 6, Smooth Muscle) and distinct clusters of cells with strong FOXA2 expression (FIG. 6, FOXA2). These results confirm that the hESCs cultivated in CA scaffolds are capable of differentiation into mesodermal tissue. A distinct group of cells with glucagons expression indicates the presence of endodermal tissue (FIG. 6, Glucagon). NCAM staining is localized within the cell distributed area, positively showing the ectoderm formation (FIG. 6, NCAM).

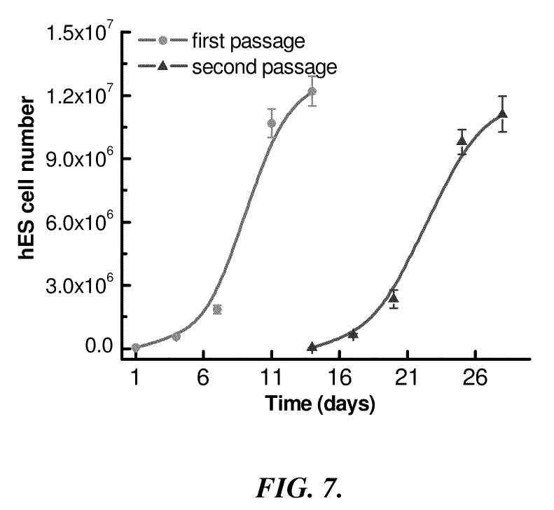

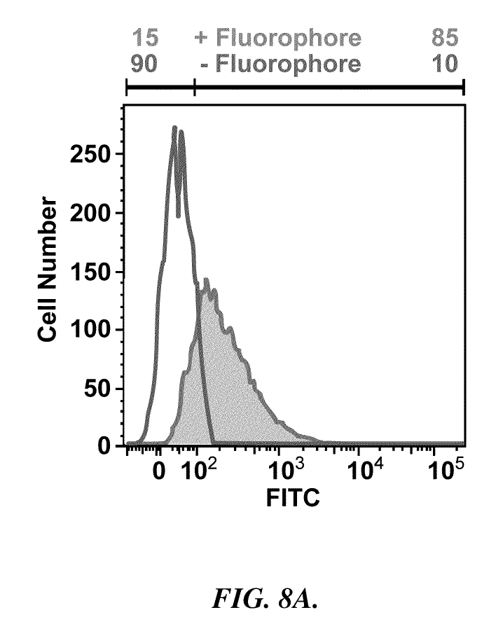

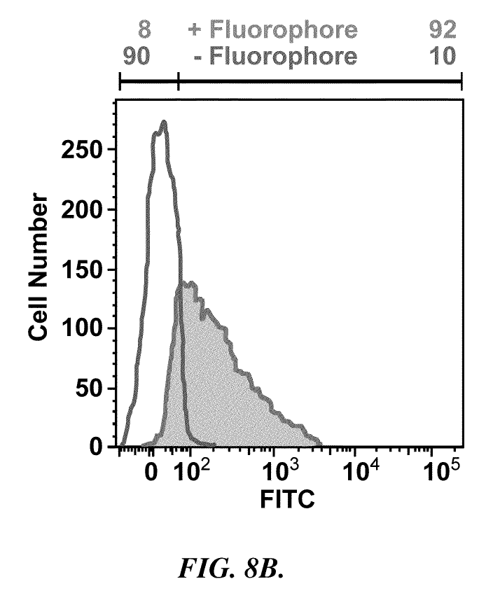

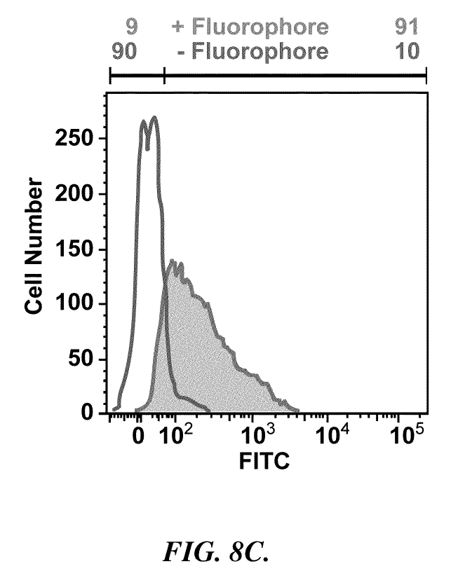

hESC recovery and subculture. To further explore the capability of the present method in renewal of a large number of undifferentiated hESCs from CA scaffolds, the renewed cells were subjected to subculture and the subcultured cells were assessed for pluripotency. Specifically, CA scaffolds seeded with an initial population of 50,000 hESCs were cultured for 14 days, and then dissolved in a solution of 100 mM EDTA and 100 mM K₂HPO₄ at room temperature. The harvested cells were then subcultured on new CA scaffolds for another 14 days. At the end of each 14-day period, hESCs were counted, cell viability was assessed by the Trypan Blue assay, SSEA4 expression was analyzed by flow cytometry analysis, and gene transcription analysis was examined by real-time PCR (RT-PCR). Cell proliferation profiles over two subsequent 14 days are shown in FIG. 7. Proliferation behavior for the second 14 days (subculture) is consistent with the one in the first 14 days. Cell recovery yield, the number of cells harvested from the CA scaffold divided by the total number of cells in the CA was determined by alamarBlue. The cell recovery yields at 14 and 28 days were 85%±2.3 and 88%±2.9, respectively. Cell viability of the hESCs recovered after both procedures exceeded 95%. Additionally, SSEA 4 expression by hESCs culture in CA scaffolds measured by flow cytometry analysis at both intervals was found to be consistently greater than that observed on hFF (FIGS. 8A-8C). The gene expression profiles of hESCs both subcultured in and released from CA scaffolds (data not shown) were in agreement with those in FIG. 3.

The present invention provides a method for sustained self-renewal of hESCs in a three-dimensional porous natural polymer scaffold (e.g., chitosan-alginate) without the support of feeder cells or conditioned medium. The pluripotency of the renewed hESCs was evaluated in vitro by evaluation of cellular proliferation, functionality, and gene activities for 21 days, and in vivo by implantation of the stem cell populated scaffolds in an immunodeficient mouse model to induce teratoma formation. The self-renewed stem cells were readily recovered for subculture by decomposing the scaffold under mild conditions. The recovered hESCs were subcultured for 14 days and their pluripotency verified.

In the practice of the invention, porous three-dimensional matrices produced from natural polymers such as chitosan and alginate have the potential to provide a reliable, low-cost solution for functional and structural restoration of damaged or dysfunctional tissues through stem cell therapy. Unlike most other natural polymers, CA scaffold can be prepared from solutions of physiological pH and thus, growth factors can be uniformly incorporated into scaffolds during the synthetic process with less risk of denaturation. Encapsulating growth factors in the scaffold matrix provides a sustained supply of the growth factors for stem cell expansion and differentiation both in vitro and in vivo, while the release rate of the growth factor can be controlled by the degradation rate of the scaffold, which is controllable by scaffold synthesis conditions. This attribute is beneficial for expanding a large number of stem cells in bioreactors, making the reality of clinical use of stem cell therapy.

The following examples are provided for the purpose of illustrating, not limiting, the invention.

### EXAMPLES

### Example 1

### Synthesis and Characterization of a Representative Scaffold: Chitosan-Alginate

In this example, the synthesis and characterization of a representative scaffold (chitosan-alginate) useful for renewing stem cells are described.

Chitosan (M_{w} 400 kDa, 85% deacetylated) and sodium alginate (alginic acid, sodium salt) were obtained from Sigma-Aldrich and used as received. Chitosan and alginate solutions were prepared separately by dissolving 4.8 g of chitosan in 80 mL 1 N acetic acid and 4.8 g sodium alginate in 120 mL 1N NaOH, respectively. The two solutions were then mixed in a blender and stirred for 1 hr to obtain a 4.8% w/v (2.4% chitosan, 2.4% alginate) solution. The resultant solution was heated and maintained at 70° C. for 1 hr. The pH of the solution was adjusted to 7-7.4 by addition of 2N acetic acid. The solution was then placed into a 24-well plate, maintained at −20° C. for 24 hrs, and lyophilized to form scaffolds. The scaffolds were then crosslinked by immersion in 1% w/v CaCl₂ solution for 10 min, and washed with deionized water.

A representative chitosan-alginate scaffold is shown in FIGS. 1A and 1B.

Compressive Strength. Compressive mechanical modulus of CA scaffolds were tested using an Instron 4505 mechanical tester with 10 kN load cells following the guidelines in ASTM D5024-95a. The specimens were cylinders of 13 mm in diameter and 12 mm in thickness. The crosshead speed of the Instron tester was set at 0.4 mm per minute, and load was applied until the specimens were compressed to approximately 30% of their original thickness. Compressive modulus was calculated as the slope of the initial linear portion of the stress-strain curve.

The compressive modulus of a representative chitosan-alginate scaffold prepared as described above was determined to be 8.1±1.5 MPa.

Tensile Strength. Tensile modulus was evaluated using a custom-made micro-tensile testing machine. The load cell has a loading range of ±30 grams with an incremental accuracy of 0.001 grams. Scaffolds were cut in rectangular shape with a cross section of 6 mm×10 mm. The tensile modulus was calculated from the stress-strain curve.

The tensile modulus of a representative chitosan-alginate scaffold prepared as described above was determined to be 0.8±1.5 MPa.

Pore Size and Porosity. The porosity and pore size distribution of the scaffold were measured by an Auto Pore IV mercury porosimeter (Micrometritis Instrument Co., Nacross Ga.). The Washburn equation was used to calculate the pore diameter. Porosity (%), total pore volume (ml/g), total pore area (m²/g), and the pore size distribution of the scaffold were determined by measuring the volume of the mercury infused. For each measurement, cylindrical scaffolds of 3 mm in diameter and 3 mm in length were placed in a 10-mL penetrometer, subjected to a vacuum of 50 mm Hg, and infused with mercury. Samples weights were measured before and after the mercury infusion.

The pores of a representative chitosan-alginate scaffold are shown in FIG. 1B and pore size distribution is illustrated in FIG. 1C. The average pore size and porosity of a representative chitosan-alginate scaffold prepared as described above were determined to be 65 μm and 95%, respectively.

### Example 2

### Stem Cell Culture, Seeding, and Evaluation

In this example, stem cell culturing, seeding, and the evaluation of stem cells renewed using representative scaffolds (chitosan-alginate) are described.

Human embryonic stem cells (hESCs), BG01V, were maintained on irradiated human fibroblast feeder layers (ATCC USA) in ES-Dulbecco's modified eagles medium (DMEM) as defined by ATCC. ES-DMEM was prepared from a 1:1 mixture of DMEM and Ham's F-12 medium containing 1.2 g/L sodium bicarbonate, 2.5 mM L-glutamine, 15 mM HEPES and 0.5 mM sodium pyruvate supplemented with: 2.0 mM L-alanyl-L-glutamine, 0.1 mM non-essential amino acids, 0.1 mM 2-mercaptoethanol, 10 ng/ml bFGF (R&D Systems), 5% (V/V) knockout serum replacement (Invitrogen), 15% (V/V) fetal bovine serum, and 100 units/ml penicillin/streptomycin. The cultures were incubated at 37° C. in the atmosphere supplemented with 5% CO₂, with the cell culture media changed daily. A solution of 200 units/ml collagenase IV in DMEM/F12 media was used to detach hESCs from the feeder layer.

CA scaffolds were sterilized with ethylene oxide gas, and cut into discs of 2 mm in thickness and 13 mm in diameter (see FIG. 1A) and fit into 24-well tissue culture plates (Corning Life Sciences). The scaffolds were incubated for 24 hrs in hESC culture media prior to cell seeding. hESCs suspended in hESC media were then seeded directly onto the scaffolds and maintained following the cell culture protocol described above. hESCs on hFF layers as a control were sub-cultured every 6 days.

Stem Cell Proliferation Assessment. Cell proliferation was assessed using the oxidation-reduction indicator alamarBlue (Alamar BioSciences, Sacramento, Calif.). The numbers of hESCs grown in CA scaffolds and on hFF layers, with initial cell seeding of 50,000, for a period of 21 days, were measured in quadruplicate at specified time intervals. Every 6 days, hESCs cultured on hFF layers were passaged and 50,000 hESCs transferred to a fresh hFF layer. For each cell number measurement, the culture was washed with PBS, and incubated in 1 mL of ES-DMEM with 10% alamarBlue substrate for 50 min or 25 min with cell numbers up to 500,000 and 3,000,000 respectively, or with 2 mL of ES-DMEM with 10% alamarBlue substrate for 30 min or 20 min with cell numbers up to 10,000,000 and 20,000,000, respectively, at 37° C. and 5% CO₂. Absorbance of the solution was measured spectrophotometrically with a microplate reader (Versamax, Molecular Devices) at 570 and 600 nm. Calibration curves for each range of cell population generated with known numbers of hESC counted by cell counter on both CA scaffolds and hFF layers were used to quantify the number of cells in each culture.

A comparison of in vitro assessment of the proliferation and pluripotency of stem cells using a representative scaffold (chitosan-alginate) and a feeder cell layer is shown in FIG. 2A.

Alkaline Phosphatase Activity Assay. The StemTAG™ alkaline phosphatase activity assay kit (Cell Biolabs) was used to quantitatively measure the alkaline phosphatase (ALP) activities of hESCs on both hFF and scaffold constructs in triplicate at specified time intervals over 21 days. The cell constructs were rinsed with cold PBS, and hESCs were detached with collagenase IV, and lysed with Cell Lysis Buffer (Sigma). After incubation at 4° C. for 10 min, the cell suspension was centrifuged at 12,000 G for 10 min to remove cell debris, retaining the supernatant. 50 μl cell lysate was mixed with 50 μl StemTAG™ ALP activity assay substrate (Sigma) in a 96-well plate. The reaction mixture was incubated for 30 min at 37° C. in 5% CO₂, until the reaction was stopped by the addition of 50 μl stop solution (Sigma). The absorbance of the product was measured at 405 nm, compared to the absorbance of 50 μl of Cell Lysis Buffer.

A comparison of alkaline phosphatase activity of stem cells using a representative scaffold (chitosan-alginate) and a feeder cell layer is shown in FIG. 2B.

Quantitative Real Time PCR. Cell-scaffold constructs were homogenized by vortexing and passing through QIAshredder columns. Total RNAs were isolated from hESCs in CA or on hFF in triplicate using RNeasy, and 30 ng of total RNA for each sample was converted to cDNA using the QuantiTect Reverse Transcription Kit following the manufacturer's instructions (Qiagen).

SYBR Green PCR Master mix (Qiagen) was used for template amplification with a primer for each of the transcripts examined. Thermocycling for all targets were carried out in a solution of 30 μl containing 0.3 μM primers (Integrated DNA Technologies) and 4 pg cDNA from the reverse transcription reaction under following conditions: 15 seconds at 94° C., 30 s at 55° C., and 30 s at 72° C. The reaction was monitored in real time using a MiniOpticon (BioRad).

FIG. 3 is a graph comparing pluripotency of stem cells in representative scaffolds (chitosan-alginate) with stem cells grown on a feeder cell layer as control.

Scanning Electron Microscopy. Cell-scaffold constructs for SEM were fixed overnight in Karnovsky's fixative containing 2% paraformaldehyde and 2% glutaraldehyde, and dehydrated with sequential washes with 50%, 75%, 95% and 100% ethanol. The samples were then air-dried and sputter-coated with Au/PD for observation with a JEOL 7000 SEM.

FIGS. 4E and 4F are scanning electron microscopy images of stem cells grown on a representative scaffold (chitosan-alginate).

Immunocytochemistry. Cell-cultured constructs were fixed in 4% paraformaldehyde, and washed in 0.2% Tween® 20 in PBS (PBST) for 30 min. The construct was then incubated in a 1:400 dilution of mouse monoclonal antibody to SSEA4 (Abcam) in PBST for 1 hr at room temperature. Following the incubation, the construct was washed in PBS for 30 min prior to incubation in a 1:500 dilution of rabbit polyclonal anti-mouse antibody conjugated to FITC (Abcam) in PBS for 1 hr. Finally, the construct was counterstained with a 1:500 solution of DAPI in PBS for 10 min. The construct was then rinsed with and maintained in PBS. Microscopy analysis was performed using a Zeiss 510 Zeta Microscope (Carl Zeiss).

FIGS. 4A-4D are microscope images assessing immunological detection of SSEA 4 and cell morphology of stem cells grown on a representative scaffold (chitosan-alginate).

Flow Cytometry. 5×10⁴ hESCs were cultured on each of CA scaffolds for 21 days and hFF layers for 6 days. The cells were detached from the hFF and CA, and processed for FACS analysis to detect SSEA4 positive cells. Mouse anti-human SSEA4 antibody (Abcam) and FITC labeled rabbit anti-mouse secondary antibody (Abcam) were used at 10 μg/mL in a 3% suspension of BSA (Sigma) in DPBS (Gibco). Cells were analyzed with a BD FACSCanto flow cytometer (Becton Dickinson Biosciences).

FIGS. 4G and 4H compare the SSEA 4 activity of stem cells harvested from representative scaffolds (chitosan-alginate) and feed cell layers.

Stem Cell Recovery and Subculture. To recover hESCs cultured on CA scaffolds, the cell-scaffold constructs were chemically decomposed and mechanically separated from hESCs. First, the constructs were rinsed gently in hESC media to remove non-adherent cells. The constructs were then decomposed in 10 mL of 100 mM EDTA and 100 mM K₂HPO₄ solution at room temperature for 5 minutes, with gentle homogenization using a syringe plunger. The resultant suspension was then forced through a 100 μm pore ceramic frit (GE Healthcare) to remove scaffold debris. Finally, cells were collected by centrifugation at 200 G for 5 min, and resuspended in cell culture media. Cell viability and recovery efficiency were determined by the Trypan Blue exclusion assay (Gibco) and hemocytomer. To evaluate the proliferation of hES cells recovered from CA scaffolds, cells were serially cultured on CA scaffolds, recovered, and subcultured again on CA scaffolds. 50,000 hES cells were seeded in quadruplicate onto 13 mm diameter×2 mm height CA scaffolds and cultured for 14 days as described in Cell culture and seeding. Proliferation was evaluated by alamarBlue (Alamar BioSciences, Sacramento, Calif.) as described above. After 14 days, cells were recovered and recovery efficiency determined. These recovered cells were then subcultured onto new CA scaffolds for an additional 14 days with proliferation and recovery assessed again.

Animal Surgery, Histology, and Immunochemistry. Ten healthy SCID nude mice (Jackson Laboratories), weighing between 25-30 grams, were hosted by the University of Washington Department of Comparative Medicine. Anesthesia was induced by ketamine/xylazine, and a 3×5×5 mm piece of the hESC-scaffold construct cultured for 21 days was inserted into the peritoneal cavity. The inserted construct was harvested one month later after euthanasia with CO₂ gas.

The explants were preserved in 4% paraformaldehyde, fixed in paraffin wax, sectioned at 5 μm, and affixed onto glass slides. One set of tissue sections were stained with either Von Kossa, or Picrosirius, or silver per standard procedures for histological analysis. Another set of tissue sections were de-waxed by 3 xylene washes, followed by xylene removal with methanol, and rinsed with cold water. Antigen retrieval was performed by boiling the de-waxed sections in 20 mM sodium citrate buffer, pH 6.0, for 15 min. The sections were then rinsed in cold water for 10 min to allow the antigen sites to reform. The sections were permeabilized with 0.025% Triton X-100 in PBS at room temperature for 10 min, and blocked with a solution of 10% rabbit serum and 1% BSA in PBS for 2 hrs at room temperature to prevent cross reaction of the secondary antibody with endogenous immunoglobulins in the tissue. The sections were then incubated with various mouse monoclonal primary antibodies in TBS with 1% BSA at 4° C. for 18 hrs. The primary antibodies (Abcam) against neural cell adhesion molecule (NCAM; 1:50), cardiac Troponin T (cTnT; 1:1), forkhead box 2 (FOXA2; 1:2000), alpha smooth muscle actin (α-SMA; 1:50), and glucagon (1:50) were used.

Following incubation with the primary antibodies, the sections were rinsed twice with 0.025% Triton X-100 in PBS for 5 min, and incubated for 2 hrs at room temperature in rabbit anti-mouse IgG secondary antibody conjugated to either FITC or rhodamine fluorophores (Abcam) at 1:500 dilution in PBS. The sections were rinsed gently twice in PBS solution, and counterstained in a 1:500 solution of DAPI in 0.025% Triton X-100 in PBS (Abcam) for 30 min. The sections were then rinsed with and maintained in PBS until analyzed using a Zeiss 510 Zeta Microscope (Carl Zeiss).

Histological analysis of tetromas retrieved after one month implantation of stem cell-scaffold contracts in SCID mice is shown in FIGS. 5A-5F. Assessment of the in vivo pluripotency of stem cells cultured in representative scaffolds (chitosan-alginate) is shown in FIG. 6.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for expanding a population of stem cells, comprising:
(a) seeding a porous scaffold comprising chitosan with stem cells; and
(b) renewing the stem cells in the presence of the scaffold to provide a scaffold populated with renewed stem cells.

2. The method of claim 1, wherein the stem cells are selected from the group consisting of embryonic stem cells and non-embryonic stem cells.

3. The method of claim 1, wherein the stem cells are selected from the group consisting of hematopoietic stem cells, bone marrow stem cells, neural stem cells, epithelial stem cells, skin stem cells, muscle stem cells, and adipose stem cells.

4. The method of claim 1, wherein the stem cells selected from the group consisting of are human stem cells, mouse stem cells, and rat stem cells.

5. The method of claim 1, wherein the renewed stem cells are substantially undifferentiated.

6. The method of claim 1, wherein the scaffold further comprises alginate.

7. The method of claim 1, wherein the scaffold further comprises a fibrous protein.

8. The method of claim 1, wherein the scaffold comprises chitosan ionically linked to alginate further crosslinked with divalent metal cations.

9. The method of claim 1, wherein the scaffold further comprises one or more growth factors.

10. The method of claim 1, wherein renewing the stem cells comprises renewing in the absence of feeder cells.

11. The method of claim 1, wherein renewing the stem cells comprises renewing in the absence of conditioned media.

12. A porous scaffold comprising chitosan populated with renewed stem cells prepared by the method of claim 1.

13. A method for administering stem cells to a subject, comprising introducing the porous scaffold of claim 12 into a subject in need thereof.

14. The method of claim 13, wherein the stem cells are selected from the group consisting of hematopoietic stem cells, bone marrow stem cells, neural stem cells, epithelial stem cells, skin stem cells, muscle stem cells, and adipose stem cells.

15. The method of claim 13, wherein the subject is a human.

16. A method for tissue engineering, comprising introducing the porous scaffold of claim 12 into a tissue to be engineered.

17. A method for treating a condition treatable by the administration of stem cells, comprising introducing the porous scaffold of claim 12 into a subject in need thereof.

18. The method of claim 17, wherein the subject in need thereof suffers from a condition selected from the group consisting of diabetes, heart diseases, Parkinson's disease, Alzheimer's disease, arthritis, leukemias, lymphomas, and bone marrow failure disorders.

19. A method for treating a condition treatable by the administration of stem cells, comprising:
(a) seeding a porous scaffold comprising chitosan with stem cells;
(b) renewing the stem cells in the presence of the scaffold to provide a scaffold populated with renewed stem cells;
(c) collecting at least a portion of the renewed stem cells from the scaffold; and
(d) administering renewed stem cells collected from the scaffold to a subject in need thereof.

20. The method of claim 19, wherein the subject in need thereof suffers from a condition selected from the group consisting of diabetes, heart diseases, Parkinson's disease, Alzheimer's disease, arthritis, leukemias, lymphomas, and bone marrow failure disorders.


SEQUENCE LISTING (28 sequences)

SEQ ID NO: 1 - DNA, 25 bp, Homo sapiens
cttgctgcagaagtgggtggaggaa

SEQ ID NO: 2 - DNA, 21 bp, Homo sapiens
ctgcagtgtgggtttcgggca

SEQ ID NO: 3 - DNA, 20 bp, Homo sapiens
cctgaacctcagctacaaac

SEQ ID NO: 4 - DNA, 20 bp, Homo sapiens
tgccacctcttagatttcat

SEQ ID NO: 5 - DNA, 20 bp, Homo sapiens
cggaagagtgtctggagcaa

SEQ ID NO: 6 - DNA, 19 bp, Homo sapiens
ggatgaagcggagtctgga

SEQ ID NO: 7 - DNA, 24 bp, Homo sapiens
caggaatttgctcgtccatctcgg

SEQ ID NO: 8 - DNA, 24 bp, Homo sapiens
tagtacgtgcagacggtggtagtt

SEQ ID NO: 9 - DNA, 20 bp, Homo sapiens
tgaaagcccacatcctaacg

SEQ ID NO: 10 - DNA, 21 bp, Homo sapiens
caagctatcctcctgctttgg

SEQ ID NO: 11 - DNA, 20 bp, Homo sapiens
tgttgcagggaagtcttact

SEQ ID NO: 12 - DNA, 20 bp, Homo sapiens
atggttttacaccgagtcac

SEQ ID NO: 13 - DNA, 20 bp, Homo sapiens
aagccacaaataacagagga

SEQ ID NO: 14 - DNA, 20 bp, Homo sapiens
gtcttctcttcccctgaagt

SEQ ID NO: 15 - DNA, 29 bp, Homo sapiens
ggatctggcagcgccgcgaagacgagcgg

SEQ ID NO: 16 - DNA, 21 bp, Homo sapiens
ttttcccatccattgtgggac

SEQ ID NO: 17 - DNA, 20 bp, Homo sapiens
accacagtccatgccatcac

SEQ ID NO: 18 - DNA, 20 bp, Homo sapiens
ttcaccaccctgttgctgta

SEQ ID NO: 19 - DNA, 20 bp, Homo sapiens
tgtggcatccacgaaactac

SEQ ID NO: 20 - DNA, 22 bp, Homo sapiens
ggagcaatgatcttgatcttca

SEQ ID NO: 21 - DNA, 20 bp, Homo sapiens
aggcgctgattgaggctcac

SEQ ID NO: 22 - DNA, 19 bp, Homo sapiens
atagatgctctgccacagc

SEQ ID NO: 23 - DNA, 20 bp, Homo sapiens
caaaaaggtggataagaacg

SEQ ID NO: 24 - DNA, 20 bp, Homo sapiens
caggtaagagtgacctgctc

SEQ ID NO: 25 - DNA, 19 bp, Homo sapiens
atgcaccgctacgacgtga

SEQ ID NO: 26 - DNA, 20 bp, Homo sapiens
cttttgcacccctcccattt

SEQ ID NO: 27 - DNA, 20 bp, Homo sapiens
ttagttgcgttacacccttt

SEQ ID NO: 28 - DNA, 20 bp, Homo sapiens
aatgtgcaatcaaagtcctc